(19) United States
(12) Patent Application Publication
(10) Pub. No.: US 2011/0281802 A1
(43) Pub. Date: Nov. 17, 2011

(54) COMPOSITION FOR TREATMENT AND PREVENTION OF HAIR LOSS AND PREMATURE GRAYING OF HAIR

(75) Inventors: Franz Paul Armbruster, Bensheim (DE); Ralf Paus, Lubeck (DE); Dorothee Langan, Osnabruck (DE)

(73) Assignee: Immudiagnostik AG, Bensheim (DE)

(21) Appl. No.: 12/745,247

(22) PCT Filed: Dec. 1, 2008
(86) PCT No.: PCT/EP2008/066542

§ 371 Date: Aug. 27, 2010

(30) Foreign Application Priority Data
Nov. 30, 2007 (EP) 07122054.5

(51) Int. Cl. A61K 38/08; G01N 33/53; A61Q 5/00; C07K 16/26; A61K 38/22; A61Q 7/00

(52) U.S. Cl. 514/12.9; 514/9.7; 530/389.2; 435/7.1

(57) ABSTRACT

Composition for treatment and prevention of human hair loss and/or hair greying, comprising an effective amount of thymic peptides of the family thymulin, thymosin alpha-1 and thymosin beta-4 and pharmaceutical excipient, diluent or carrier. Method and pharmaceutical composition for treatment and prevention of balding, hair loss, and alopecia.

### FIELD OF THE INVENTION

The invention relates to compositions for treatment and prevention of hair loss and premature greying of hair.

### BACKGROUND OF THE INVENTION

The thymus was not a well-understood organ until studies in the 1960's that animals whose thymus organs were removed, developed profound immune deficiencies and died. Ever since the thymus is known to be responsible for the development and regulation of T cell immunity. The thymus seems to exert its regulatory functions through the secretion of various non-cellular, hormone-like products, called thymic peptides.

Thymulin is a nonapeptide, which is active when coupled with zinc and which plasma levels follow a circadian rhythm, peaking in the early hours of the morning (Safieh-Garabedian et al., Thymulin and its role in immunomodulation; J. Autoimmunity 1992, 5:547-555; Mocchegiani et al., Presence of links between zinc and melatonin during the circadian cycle in old mice: effects on thymic endocrine activity and on survival; J. Neuroimmunology 1998, 86:111-122). Recent research showed that thymulin acts favourably in patients with viral diseases, chronic pain and cancer. Thus, thymulin is suspected to play a role in the communication between the neuroendocrine system and the immune system. Thymulin also affects the release of ACTH, PRL, GH, TSH and LH from the pituitary gland (Hadley et al., Thymulin stimulates corticotrophin release and cyclic nucleotide formation in the rat anterior pituitary gland; Neuroimmunomodulation 1997, 4:62-9; Brown O A et al., Thymulin stimulates prolactin and thyrotropin release in an age-related manner; Mechanisms of Aging and Development 1998, 104: 249-62; Brown O A et al, Growth hormone-releasing activity of thymulin on pituitary somatotropes is age dependent; Neuroendocrinology 1999, 69:20-27; Goya R G et al, Thymulin and the neuroendocrine system, Peptides 2004, 25:139-142). However, there is evidence that pituitary hormones themselves regulate thymulin secretion (Dardenne et al., Neuroendocrine control of thymic hormonal production. Prolactin stimulates in vivo and in vitro the production of thymulin by human and murine thymic epithelial cells, Endocrinology 1989, 125: 3-12).

Thymosin alpha-1 (TA1) is a 28aa peptide, which modulates lymphocyte differentiation and is involved in stimulating the production of antibodies and cytokines; endothelial cell migration, angiogenesis and wound healing (Cordero et al., Thymic Peptides and Preparations: an Update; Arch Immun Therap Exp 1999, 47:77-82; Malinda KM et al, Thymosin alpha 1 stimulates endothelial cell migration, angiogenesis and wound healing; J Immunol 1998, 160:1001-6). Further proposed therapeutic applications encompass the enhancement of resistance to infections and the treatment of viral infections and cancer (Hannappel et al, The Thymosins: Prothymosin alpha, parathymosin, and beta-thymosins: structure and function; Vitamins and Hormones 2003, 66:257-296).

Thymosin beta-4 (TB4) occurs in most human cells at high concentrations, with the highest concentration in platelets. Erythrocytes contain no thymosin β4 (Huff T et al., beta-Thymosins, small acidic peptides with multiple functions; Intern. J Biochem & Cell Biol 2001, 33: 205-220). Numerous biological effects have been reported for this peptide but best known are its inhibition of G-actin polymerization and the stimulation of cell migration (Huff T et al., beta-thymosins, small acidic peptides with multiple functions; Intern. J Biochem & Cell Biol 2001, 33: 205-220). Thymosin beta-4 has been found to stimulate wound healing, decrease inflammation reactions and increase re-epithelialisation and angiogenesis (Malinda K M et al., Thymosin beta4 accelerates wound healing; J Invest Dermatol. 1999, 113:364-8, Philp D et al, Thymosin beta4 promotes angiogenesis, wound healing, and hair follicle development, Mech Aging and Develop 2004, 125:113-115). Thymosin beta-4 is expressed in hair follicle stem cells in a cycle-related fashion and was noted to stimulate hair growth in rats (Philp D et al, Thymosin beta4 increases hair growth by activation of hair follicle stem cells; The FASEB J Express Article doi:10.1096/fj.03-0244fje published online Dec. 4, 2003). WO 03/063775 discloses the use of thymosin beta-4 and peptides thereof for the promotion of hair growth and treatment of alopecia.

It is clear that thymic peptides are powerful biological mediators. Notwithstanding, the full extent of effects of thymic peptides is still not known. Consequently, the state of the art represents itself as a problem and it is an object of the invention to provide further compositions and therapeutic applications on the basis of thymic peptides.

### SUMMARY OF THE INVENTION

The present invention provides a pharmaceutical composition for treatment and prevention of hair loss and premature human hair greying, comprising an effective amount of thymic peptides, in particular thymulin, thymosin alpha-1 (TA1) and thymosin beta-4 (TB4).

In one embodiment of the invention, the pharmaceutical composition comprises an effective amount of thymulin and/or thymosin alpha-1 for treatment of hair shedding and telogen effluvium by catagen inhibition. Further applications of the pharmaceutical composition are for treating chronic telogen effluvium, nonscarring alopecia, alopecia, hair loss, acute hair loss, non-scarring alopecia, balding.

In another embodiment of the invention there is provided a pharmaceutical composition comprising an effective amount of thymulin for stimulating human hair follicle pigmentation and/or melanin granule production.

In still further embodiments, the present disclosure provides a pharmaceutical composition comprising effective amounts of thymulin and/or thymosin alpha-1 for maintaining hair follicle cells longer in the anagen phase of the hair cycle.

In preferred embodiments, the present disclosure contemplates that the effective amount of thymic peptides is in a formulation for topical application since such an application usually results in less systemic side effects. A further preferred embodiment contemplates a composition wherein the effective amount of thymic peptides is in a formulation for oral administration. In case of psychological strain, an intravenous application of the formulation is contemplated. The effective concentrations and a preferred dosage can be taken from the examples, while a preferred dosage has still to be found. It is contemplated that the thymic peptides of the invention must be present in the composition in a concentration for topical application in an amount from of 1 pg to 1000 ng/mL, preferably 10 pg/ml to 100 ng/ml. Compositions for oral administration or by injection, intravenous, subcutaneous, intramuscular or intra-peritoneal, will contain the thymic peptides for treatment of telogen effluvium, balding, hair loss, alopecia in a dosage for administration in a range from 1 pMol to 1 nmol/kg.

### BRIEF DESCRIPTION OF THE DRAWINGS

FIG. 1 is a schematic representation of the experimental design of assay #1;

FIG. 2 is a schematic representation of the experimental design of assay #2;

FIG. 3 is a schematic representation of the experimental design of assay #3;

FIGS. 4A-4C are graphs showing the mean elongation in percent compared to day 0 effected by various concentrations of thymulin, TA1 and TB4 in assay #1;

FIG. 5 is a graph showing the mean elongation in percent compared to day 0 effected by thymulin and TB4 in assay #2;

FIGS. 6A-6C are graphs showing the mean elongation in percent compared to day 0 effected by thymulin, TA1, and TB4 in assay #3;

FIG. 7 is a diagram showing the mean relative darkening of the groups measured by image J obtained by a treatment with thymulin, TA1, and TB4;

FIGS. 8A-8C are diagrams showing the proportion of hair follicles in different stages of the hair cycles for various hair follicle preparations in assays #1 and 3;

FIG. 9 is a diagram showing the mean percentage of Ki67-positive cells and the standard error of the measurement (SEM);

FIG. 10 is a digital image on a photomicrograph showing a immunohistological staining of human dermis (scalp skin) and epidermis using anti-thymulin antibodies (dilution 1:1000), 100-fold enlargement;

FIG. 11 is a digital image on a photomicrograph showing a immunohistological staining of a human hair follicle (epidermis close to basal membrane) using anti-thymulin antibodies (dilution 1:1000), 100-fold enlargement;

FIG. 12 is a digital image on a photomicrograph showing a immunohistological staining of a hair shaft using anti-thymulin antibodies (dilution 1:1000), 100-fold enlargement;

FIG. 13 is a digital image on a photomicrograph showing a immunohistological staining of human dermis and epidermis (medulla, cortex and outer root sheath) using anti-thymosin-alpha-1 antibodies (dilution 1:500), 100-fold enlargement;

FIG. 14 is a digital image on a photomicrograph showing a immunohistological staining of a human hair follicle using anti-thymosin-alpha1 antibodies (dilution 1.500), 100-fold enlargement;

FIG. 15 is a digital image on a photomicrograph showing a immunohistological staining of the human hair shaft (inner root sheath) using anti-thymosin alpha 1 antibodies (dilution 1:500), 100-fold enlargement;

FIG. 16 is a digital image on a photomicrograph showing a immunohistological staining of the human dermis and epidermis (outer root sheath) using anti-thymosin beta-4 antibodies (dilution 1:500), 100-fold enlargement;

FIG. 17 is a digital image on a photomicrograph showing a immunohistological staining of epidermis and dermis (only partially) close to basal membrane using anti-thymosin beta-4 antibodies (dilution 1:500), 200-fold enlargement;

FIG. 18 is a digital image on a photomicrograph showing a immunohistological staining of a human hair follicle (cytoplasm) using anti-thymosin beta-4 antibodies (dilution 1:500), 100-fold enlargement;

FIG. 19 is a digital image on a photomicrograph showing a immunohistological staining of the proximal part of a human hair follicle (the matrix cells in the pulp) using anti-thymosin beta-4 antibodies (dilution 1:500), 200-fold enlargement;

FIGS. 20a,b are digital images on photomicrographs showing a immunohistological staining of hair follicles for prolactin;

FIGS. 21a,b are digital images on photomicrographs showing a immunohistochemical staining of hair follicles for prolactin when treated with 100 pg/mL (a) and 10 ng/mL (b) thymulin, respectively;

FIG. 22A is a digital image on a photomicrograph showing a immunohistological staining of a human hair shaft after acetone fixation using a rabbit polyclonal anti-thymosin antibody (dilution 1:100, lmmundiagnostik Ak 9510), 40-fold enlargement;

FIG. 22B is a digital image on a photomicrograph showing a immunohistological staining of a human hair shaft (root sheath) after acetone fixation using a rabbit polyclonal anti-thymosin antibody aa 75-109 (dilution 1:1000, Immundiagnostik Ak 9540), 40-fold enlargement;

FIG. 22C is a digital image on a photomicrograph showing a immunohistological staining of a human hair shaft (root sheath) after acetone fixation using a rabbit polyclonal anti-thymosin antibody aa 101-109 (dilution 1:200, Immundiagnostik Ak 9540), 40-fold enlargement;

FIGS. 23A,B are digital images on photomicrographs showing a immunohistological staining of human dermis and epidermis (scalp skin) and of a hair follicle using polyclonal rabbit anti-human thymosin antibodies (dilution 1:100), 100-fold enlargement, and a corresponding control.

### DETAILED DESCRIPTION OF THE INVENTION

The present disclosure relates to methods and compositions for treatment and prevention of human hair loss and/or hair greying. In particular, the present disclosure provides composition comprising an effective amount of thymic peptides of the family thymulin, thymosin alpha-1 and thymosin beta-4 and a pharmaceutical excipient, diluent or carrier. In some preferred embodiments, the present disclosure provides pharmaceutical composition comprising effective amounts of thymulin and/or thymosin alpha-1 for maintaining hair follicle cells longer in the anagen phase of the hair cycle, and in particular, for treating hair shedding and telogen effluvium by catagen inhibition. The disclosure therefore encompasses pharmaceutical compositions comprising thymulin and TA1 for treatment of chronic telogen effluvium, nonscarring alopecia, alopecia, hair loss, acute hair loss, non-scarring alopecia, balding. In some preferred embodiments, the disclosure provides pharmaceutical composition comprising an effective amount of thymulin for stimulating human hair follicle pigmentation and/or melanin granule production.

While thymosin-beta and topically applied thymus extracts have long been claimed to stimulate hair growth in rats, we note that it was never proven that they can stimulate human scalp hair growth. Therefore, we have explored whether thymic peptides alter human hair growth and/or pigmentation in vitro. Instead, we have found that all tested thymic peptides thymulin, Thymosin alpha-1 [TA1], Thymosin-beta4 [TB4]) significantly inhibited hair shaft production of organ-cultured human scalp hair follicles (HF) under serum-free conditions. However, thymulin or TA1-treated hair follicles stayed longer in anagen than vehicle-treated control hair follicles. Nevertheless, TA1-treated hair follicles showed fewer proliferating hair matrix keratinocytes than control hair follicles. Quantitative Masson-Fontana histochemistry revealed further that thymulin-treated hair follicles had significantly more melanin granules than controls. TA1 and TB4 did not influence HF melanogenesis. Immunohistochemistry for all three thymic peptides appeared to show specific immunoreactivity in human scalp skin sections. Thymulin and TA1 follow similar patterns with strongest immunoreactivity seen in epidermis and in the HF inner root sheath (especially Henle's layer and medulla, distal to the hair bulb). TB4 immunoreactivity was much more widespread, and included also outer root sheath, dermal sheath and dermal papilla of the HF. Only hair matrix keratinocytes appeared to be negative for all three thymic peptides. Furthermore, we disclose that thymulin stimulates human hair follicle pigmentation. These combined data here provide first definitive evidence that thymic peptides alter human hair follicle biology, and suggest that thymulin and TA1 reduce telogen effluvium by catagen inhibition, even though actual hair shaft formation is also inhibited.

Telogen effluvium is a form of nonscarring alopecia characterized by diffuse hair shedding, often with an acute onset. Telogen effluvium can affect hair on all parts of the body, but, generally, only loss of scalp hair is symptomatic. Understanding the pathophysiology of telogen effluvium requires knowledge of the hair growth cycle. All hair has a growth phase, termed anagen, apoptosis-driven regression (catagen) and a resting quiescent phase, telogen (Paus et al., The biology of hair follicles, NEJM 1999, 341:491-497). On the scalp, anagen lasts approximately 3 years, while telogen lasts roughly 3 months, although there can be wide variation between individuals. During telogen, the resting hair remains in the follicle until it is pushed out by growth of a new anagen hair. In most people, 5-15% of the hair on the scalp is in telogen at any given time. Shedding does not occur until the new anagen hairs begin to grow. The emerging hairs help to force the resting hairs out of the follicle. Recent evidence suggests that the mechanism of shedding of a telogen hair is an active process that may occur independent of the emerging anagen hair.

The symptom of both acute and chronic telogen effluvium is increased hair shedding and diffuse hair loss from the entire scalp. Acute telogen effluvium is defined as hair shedding lasting less than 6 months. Patients usually only complain that their hair is falling out at an increased rate or that the remaining hair feels less dense. Causes for telogen effluvium and acute hair shedding can be physiologic stress, papulosquamous diseases of the scalp such as psoriasis and seborrheic dermatitis, allergic contact dermatitis, immunizations, severe infections (HIV), acute illness such as febrile illness, major surgery and severe trauma as well as chronic illness such as malignancy, particularly lymphoproliferative malignancy, systemic lupus erythematosus, end-stage renal disease, or liver disease, hormonal changes such as pregnancy and delivery (can affect both mother and child), hypothyroidism, discontinuation of estrogen-containing medications; changes in diet like crash dieting, anorexia, low protein intake, and chronic iron deficiency, heavy metals such as selenium, arsenic, and thallium. Acute telogen effluvium can occur in either sex, but because hormonal changes in the postpartum period are a common cause of telogen effluvium, women may have a greater tendency to experience this condition. In addition, women tend to find the hair shedding more troublesome than men do so that more women seek medical attention for this condition. Patients with acute telogen effluvium usually complain of relatively sudden onset of hair loss. If greater than 25% of extracted hairs are in telogen, the diagnosis of telogen effluvium is confirmed. However, each patient's scalp hair has an individual characteristic growth cycle. There are patients who have a very long anagen phase and a small proportion of hair in telogen at any given time. Medications, of which the most frequency cited are beta-blockers, anticoagulants, retinoids (including excess vitamin A), propylthiouracil (induces hypothyroidism), carbamazepine, and immunizations. Thus, this type of medication represents itself as a problem.

The hair is composed of a protein called keratin. The hair itself is arranged in three layers, an outer cuticle, middle cortex and central medulla. If the hair is coloured it is due to the presence of pigments—either melanin (black or brown) or pheomelanin (red or yellow). If these pigments are lacking the hair is white. Canites is the term given to grey hair, it is an illusion created by the mixture of white and coloured hairs. Hair grows from a follicle. The walls of the follicle form the outer root sheath of the hair. The lower part of the follicle widens out to form the hair bulb that contains the germinal matrix, the source of hair growth. Dermal tissue projects into the follicle base to form the dermal papilla, and this has a network of capillary blood vessels to supply oxygen, energy and the amino acids needed for growth. Melanocytes are present in the upper part of the papilla, producing pigment granules that are distributed throughout the cortex. In the follicle an inner root sheath that has three layers surrounds the hair. The Henle's layer is one cell thick and lies to the outer root sheath. Huxley's layer is two or three cells thick and is in the middle of the sheath. The cuticle of this inner root sheath interlocks with the cuticle of the hair. Both the hair and the inner root sheath grow at the same rate, but the inner root sheath breaks down about two-thirds of the way up the follicle, so only the hair emerges past the skin surface. Uncut hairs have a pointed tip.

The present invention provides the first time a pharmaceutical composition that has immediate effect on the hair shedding. While not promoting hair growth, the tested thymic peptides (thymulin, thymosin alpha-1, thymosin beta-4) significantly inhibited hair shaft production of organ-cultured human scalp hair follicles (HF) under serum-free conditions, while interindividual variations were evident as expected, and most interestingly, thymulin and/or TA1-treated hair follicles stayed longer in anagen phase than vehicle-treated control hair follicles. Thus, thymic peptides can be used as an effective ingredient in a pharmaceutical composition to inhibit telogen effluvium and hair shedding in both men and women. Most importantly, it has further been found that the application of thymulin leads to increased pigmentation and melanin granule production and is consequently an active ingredient against premature greying of hair.

In one embodiment, the present disclosure provides an easy to use topical therapeutic composition and treatment for avoiding loss of hair.

In another embodiment, the present disclosure provides a therapeutic composition for topical application for increasing the lustre of hair as well as decreasing the greying of hair.

A further embodiment in the present disclosure is represented by a treatment for hair loss through topical application of thymic peptides in various formulations designed for topical application.

Another embodiment is represented by a treatment for hair loss through the oral application of thymic peptides in various formulations designed for oral administration.

Another aspect of the invention is represented by a treatment for hair loss and premature hair greying through the oral application of thymic peptides, notably thymulin and TA1 in various formulations designed for oral application, optionally combined with co-enzyme Q and acetyl carnitine.

Still another aspect of the invention is represented by a composition and treatment for hair loss and hair greying through the topical application of thymic peptides, notably thymulin and TA1 in various formulations designed for topical application, optionally combined with co-enzyme Q and acetyl carnitine in various formulations designed for topical application.

The diagnostic aspect of the present invention is represented by the use of antibodies against thymulin, TA1 and TB4 in a composition for diagnosis of telogen effluvium, upcoming alopecia, balding and hair loss.

### EXAMPLES

This invention has been achieved by investigating the effects of thymosin alpha-1 (TA1), thymosin beta-4 (TB4) and thymulin on human hair follicles (HF) in vitro and by studying and assessing the following parameters: elongation of hairshaft, melanogenesis/pigmentation, percentage of cells proliferating and apoptosis and percentage of cells in various hair cycle stages. The results have further been confirmed by investigating and assessing the amount and distribution of thymic peptides in human scalp skin and human hair follicles, namely by immunohistochemistry and ELISA.

### Example 1

### I. Patients

Human anagen VI hair follicles were isolated from scalp skin obtained from three different individuals undergoing routine face-lift surgery. All experiments were performed according to Helsinki guidelines.

TABLE 1
Details of patients

| | Name of | | | Age |
|---|---|---|---|---|
| | sample | Sex | Localisation | (years) |
| Assay #1 | HF 429 | f | occipital | 69J. |
| Assay #2 | HF 441 | f | occipital | 44J. |
| Assay #3, #5a, #5b and | HF 446 | f | occipital | 61J |
| Assay #4 | HS 336 | m | temporal | 58J. |
| Assay #5c | HS 366 | f | temporal | 58J |

### II. Isolation and Maintenance of Human Hair Follicles (Culture)

Hair follicles were isolated as described (Philpott MP et al, Human hair growth in vitro; J Cell Science 1990, 97(Pt 3):463-71). Isolated HFs were maintained in supplemented, serum-free Williams E medium (Biochrom, Cambridge, UK) supplemented with 2 mMol/L L-glutamine (Invitrogen, Paisley, UK), 10 ng/mL hydrocortisone (Sigma-Aldrich, Taufkirchen, Germany), 10 μg/mL insulin (Sigma-Aldrich), and 1% antibiotic/antimycotic mixture (Gibco, Karlsruhe, Germany). Hair follicles were placed in a 24-well plate with 500 μL medium (day 0) and incubated in a CO2-incubator (5% CO2, 37° C.) over night without any treatment. The next day (day 1) medium was exchanged and test substance was added to each well (5 μL of stock solution was added to 495 μL culture medium). The medium or medium with test substance was changed every 48 hours.

### III. Experimental Design

Assay #1 Culture HF429: Hair follicles were grown in the wells of a microtiter plate (3 follicles per well) and treated for 9 days with culture medium and test substance as indicated in FIG. 1. Thereafter, elongation, melanogenesis and hair cycle stage were determined.

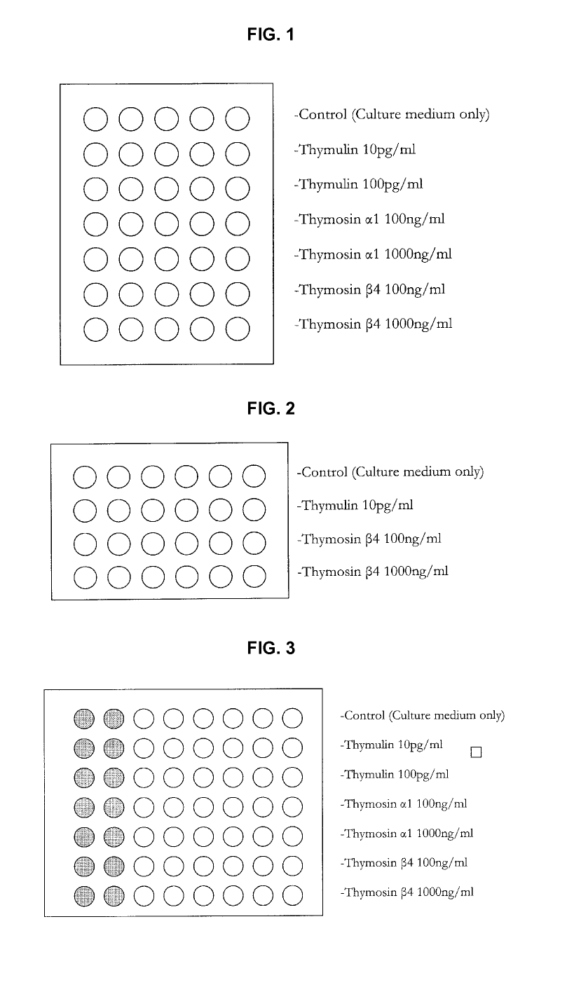

Assay #2 Culture HF 441: Hair follicle cultures (3 follicles/well) were treated for 9 days with culture medium and a combination of test substances at various concentrations as shown in FIG. 2, followed by a determination of elongation and hair cycle stage.

Assay #3 Culture HF 446: Eight hair follicle cultures (3 follicles/well) were treated with various concentration of test substance as described for 2 days, followed by snap freezing and sectioning of two wells. Six wells were treated up to 9 days, then snap frozen and sectioned. The distribution of test substances and concentrations were as indicted in FIG. 3. The parameter measured after two days was proliferation/apoptosis, and the parameters measured after 9 days were elongation, proportion of cells in proliferation/apoptosis, and hair cycle stage

Assay #4 Treatment of human scalp skin treated with antibodies against thymic peptides: Snap frozen human scalp skin sections of a 58 year old male (temporal area) were treated as follows: Inhibition of endogenous peroxidase with 3% H2O2 in TBS for 15 minutes, followed by three washings in TBS for five minutes, and pre-incubation with 10% goat serum (Dako Cytomation, Glostrup, Denmark; Cat No 50114121) in TBS for 20 minutes, incubation with primary antibody overnight at 4° C. (rabbit anti-Thymosin 1, anti-Thymosin β4, anti-Thymulin, Immundiagnostik AG, Bensheim, Germany—CatNo's A9530.2, A9510.2, A9522.2 respectively; anti-Thymosin 1 and anti-Thymosin β4 diluted 1:500 in TBS comprising 2% goat serum (Dako Cytomation). The anti-Thymulin was diluted 1:1000). A further washing in TBS followed and incubation with secondary antibody (goat anti-rabbit-biotinylated, Jackson Immunoresearch, PA, USA; CatNo 111-065-045), diluted 1:200 in TBS comprising 2% goat serum for 45 minutes at room temperature. A treatment with avidin/biotin complex-peroxidase (Vector Laboratories, Burlingame, Calif., USA; CatNo PK-6100) for 30 minutes followed and then a further washing in TBS. Thereafter treatment with AEC+substrate (Vector Laboratories, Burlingame, Calif., USA; CatNo SK-4200) for 5 minutes and washes in TBS. Counterstaining was done with Mayer's Haemalaun (Chroma Muenster, Germany; CatNo 2E038) and mounting with Aquamount (DAKO Cytomation, CatNo S3025).

Assay #5—ELISA—amount of thymic peptides in human hair follicles: Three samples were tested: Assay 5a: 20 Hair follicles from the occipital area of a 61 yr old female patient; Assay 5b: 20 Hair follicles from same location and of same female patient as in 5a; Assay 5c: 20 Hair follicles from the temporal area of a female patient (58 yr). The twenty dissected hair follicles were immersed in phosphate buffer comprising 1% (w/v) TRITON™ X (Fluka, Sigma Aldrich, Taufkirchen, Germany; CatNo 93420). 550D1 PBS was added and the samples were emulsified by ultrasonic disintegration and centrifugation. The supernatant was used in the ELISA. A sample of the diluted antigen Thymulin, Thymosin α1 and Thymosin β4 (all Immundiagnostik AG, Bensheim, Germany, CatNos A 9530AG.1, A 9510AG1 and A 9522AG.1, respectively) were used as positive control. The thymulin, thymosin α1 and thymosin β4 were determined using commercial ELISA kits (Thymulin EIA—CatNoK9810; Thymosin α1 ELISA—CatNo K9510: Thymosin β4 EIA—CatNo K9520—all Immundiagnostik AG, Bensheim, Germany).

Assay #6—Stimulation of expression of pituitary hormones by Thymulin (PRL+TSH): Hair follicles from the occipital area of a 61 year old female were cultured for 9 days and treated with culture medium and thymulin 10 pg/mL (human thymulin, No A9530AG.1; Immundiagnostik AG, Bensheim, Germany) as described above, then snap frozen and sectioned. The staining was performed according to the following protocol:

A. Prolactin: Pre-treatment with 0.5% Triton X (Fluka, Sigma Aldrich, Taufkirchen, Germany; CatNo 93420) in TBS for 10 minutes at room temperature, three washings in TBS for five minutes each, treatment with 0.6% H₂O₂ in methanol three times for 15 minutes, washes in TBS. Pre-incubation with 10% rabbit serum (Dako Cytomation, CatNo X0902) in TBS with 3% BSA for 20 minutes, incubation with primary antibody (goat anti-human prolactin, Santa Cruz Biotechnology, Inc., Santa Cruz, Calif., USA; CatNo SC-7805), dilution 1:100 in TBS+2% rabbit serum (Dako Cytomation, CatNo X0902) overnight at 4° C. Thereafter washes in TBS. Incubation with secondary antibody (rabbit anti-goat-biotinylated, DAKO Cytomation, CatNo E0466) diluted to 1:200 with 2% rabbit serum in TBS for 45 minutes at room temperature. Treatment with Avidin Biotin Complex-alkaline phosphatase (Vector Laboratories, Burlingame, Calif., USA; CatNo AK-5000) for 30 minutes, then washing in TBS. Thereafter treatment with Fast Red Tablets (Sigma-Aldrich, CatNo F4648-50SET) for 6 minutes. Counterstaining with Mayer's Haemalaun (Chroma Muenster, CatNo 2E038) and mounting with Aquamount (DAKO Cytomation, CatNo S3025).

B. TSH: Pre-incubation with 10% goat serum (Dako Cytomation, CatNo 50114121) in TBS for 20 minutes, then incubation with primary antibody (mouse anti-human TSH, Dianova, Hamburg, Germany, CatNo DLN 07830), dilution 1:20 in TBS+2% goat serum (Dako Cytomation, CatNo 50114121) overnight at 4° C. Thereafter washing in TBS. Incubation with secondary antibody (goat anti-mouse-biotinylated, Beckman Coulter, Inc. Fullerton, Calif., USA, CatNo IM0816) diluted to 1:200 with 2% goat serum in TBS for 45 minutes at room temperature. Treatment with avidin/biotin-complex coupled with alkaline phosphatase (Vector Laboratories, Burlingame, Calif., USA; CatNo AK-5000) for 30 minutes, then washes in TBS. Thereafter treatment with Fast Red Tablets (Sigma-Aldrich, CatNo F4648-50SET) for 6 minutes. Counterstaining with Mayer's Haemalaun (Chroma Muenster, CatNo 2E038) and mounting with Aquamount (DAKO Cytomation, CatNo S3025).

In both assays human pituitary gland was set as positive control and negative control were hair follicles treated without the primary antibody.

### IV. Measurement of Elongation

Hair shaft length measurements were performed every second day on individual hair follicles using a binocular microscope with an eyepiece measuring reticule. The length of the follicle was defined as the distance between the base of the bulb and the cut end of the hair fibre (Philpott MP et al, Human hair growth in vitro; J Cell Science 1990, 97(Pt 3):463-71).

### V. Measurement of Melanogenesis

Fontana Masson staining was performed at the end of the culture period and photos taken of snap frozen sections. The photos were analysed (Image J, public domain, developed at the National Institutes of Health, USA) by measuring the density of two squares, consisting of 150×150 dots, placed over the darkest areas on both sides of the dermal papilla. For Fontana-Masson staining, fixed sections were treated in darkness for 40 minutes with a solution containing 10% silver nitrate (Merck & Co., Inc., Whitehouse Station, N.J., USA; CatNo 1.01512.0100), following by a development in solution containing 5% sodium thiosulphate (Merck & Co., Inc.; CatNo 1.06512.2500) for 1 minute and multiple washings in distilled water for 3 minutes. Counterstaining with 0.5% Neutralrot (Chroma Muenster, Germany, CatNo 2E038). Then dehydration in xylol and covering with Eukitt (O. Kindler GmbH, Freiburg, Germany; CatNo E115).

### VI Assessment of Percentage of Proliferating Cells and Cells Undergoing Apoptosis

After nine days, Ki67/TUNEL staining was performed and photos taken.

Proliferating and resting cells as well as cells undergoing apoptosis were counted separately proximal Auber's line. Results are expressed as percentages. For Ki-67/TUNEL staining, fixed sections were incubated for 60 minutes in a solution comprising 30% TdT-enzyme (Chemicon International, Inc., Temecula, Calif., USA; CatNo S1707). 2 mL stop buffer (Chemicon International, Inc.; CatNo S7110), diluted in 68 mL distilled water, was added for 10 minutes to stop the reaction. This was followed by a pre-incubation for 20 minutes with 10% goat serum (Dako Cytomation, CatNo 50114121) and incubation with mouse-anti human Ki-67 antigen (Chemicon International, Inc., CatNo S7110) 1:20 in PBS with 2% goat serum. The fixed section was then washed in PBS, treated with anti-digoxigenin antibody (Chemicon International, Inc., CatNo S7110) (59DI Antibody solution and 5601 Stop solution, 30 minutes) and goat anti-mouse-IgG-rhodamine red (Jackson Immuno Research, Suffolk, UK; CatNo 115-295-062), 1:200 in PBS with 2% goat serum. After washes with PBS, counterstaining with DAPI™ (10 g/mL) (Roche, Basel, Switzerland; CatNo 236276) and further washes with PBS and mounting using Fluoromount (Southern Biotech, Birmingham, Ala., USA; CatNo 0100-01).

### VII. Assessment of Hair Cycle Stage

Photodocuments from the Fontana Masson staining were used for hair follicle staging according to previously well-defined morphological criteria (Stenn K S et al, Controls of Hair Follicle Cycling; Physiol Rev 2001, 81:449-494, Müller-Röver S et al, A Comprehensive Guide for the Accurate Classification of Murine Hair Follicles in Distinct Hair Cycle Stages; J Invest Dermatol 2001, 117:3-15). The percentage of hair follicles in anagen, early, mid and late catagen was determined.

### VIII. Statistical Analyses

All data were analysed by two-tailed unpaired t-test (SPSS analysis software SPSS Inc., Chicago, USA). For determination of elongation, two outliers at top and bottom end of value range were eliminated; with the measurement of melanogenesis one outlier at both, top and bottom range, was eliminated. Results are given as mean value and standard error of measurement (SEM).

### Results

Microdissected anagen VI hair follicles of three independent experiments were treated with medium only or thymulin, TA1 or TB4 at various concentrations. Elongation was measured every two days and growth ratio was calculated. Elongation of different groups was compared to each other and the elongation results were analysed using Student's t-test. Treatment by TA1, TB4 and thymulin gave no statistical significant prolonged hair growth. Instead, treatment with TB4 (100 ng/mL and 1000 ng/mL), TA1 (100 and 1000 ng/mL) and thymulin (100 pg/mL) lead to a significantly reduced hair growth compared with the control, while substantial interindividual differences in the HF response to thymic hormones was observed.

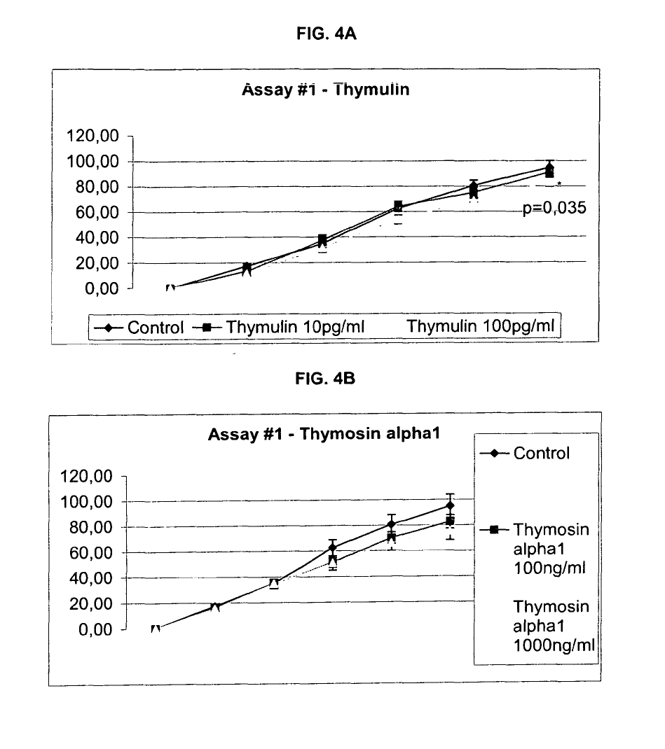

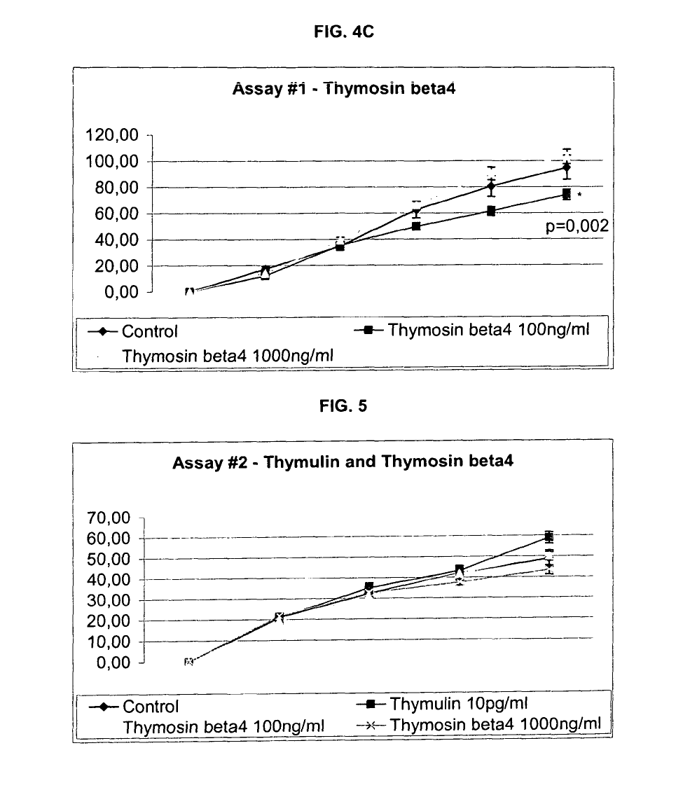

In the first experiment (assay #1) the relative elongation of the groups was between 74.11 and 103.17% at day 9. Follicles treated with thymosin β4 100 ng/mL and thymulin 100 pg/mL showed a significant lack of hair growth (p<0.05). In the second experiment (assay #2) only two substances were tested, one only in a single concentration. We were not able to detect any significant differences of relative hair growth. The elongation of the groups was between 43.30 and 58.90%. In a third experiment (assay #3) the relative elongation of the groups was between 69.90 and 94.36%. The treatment with thymosin α1 at 100 and 1000 ng/mL and thymosin β4 at 1000 ng/mL produced a significantly lowered rate of hair growth (p<0.05). The differences between the results of assays #1, #2 and #3 also point to interindividual variations in the response of human scalp hair follicles to thymic peptides. The results as to the mean elongation in percent compared with day 0 are graphically summarized in Tables 2 to 4 below and FIGS. 4A-4C

TABLE 2
Mean elongation with thymulin in percent compared to day 0

| | Day 0 | Day 1 | Day 3 | Day 5 | Day 7 | Day 9 |
|---|---|---|---|---|---|---|
| Control | 0 | 17.38 | 34.92 | 62.57 | 80.50 | 94.86 |
| Thymulin | 0 | 13.19 | 37.58 | 64.05 | 75.10 | 90.98 |
| 10 pg/mL | | | | | | |
| Thymulin | 0 | 11.33 | 31.50 | 53.75 | 68.76 | 83.13 |
| 100 pg/mL | | | | | | |

SEM:

| | | | | | | |
|---|---|---|---|---|---|---|
| Control | 0.00 | 1.86 | 2.64 | 2.55 | 4.21 | 5.02 |
| Thymulin | 0.00 | 3.17 | 4.11 | 4.13 | 4.01 | 3.36 |
| 10 pg/mL | | | | | | |
| Thymulin | 0.00 | 1.86 | 3.11 | 3.45 | 1.69 | 3.50 |
| 100 pg/mL | | | | | | |

TABLE 3
Mean elongation in percent compared to day 0:

| | Day 0 | Day 1 | Day 3 | Day 5 | Day 7 | Day 9 |
|---|---|---|---|---|---|---|
| Control | 0.00 | 17.38 | 34.92 | 62.57 | 80.50 | 94.86 |
| Thymosin alphα1 | 0.00 | 16.71 | 34.54 | 51.14 | 70.11 | 82.55 |
| 100 ng/mL | | | | | | |
| Thymosin alphα1 | 0.00 | 15.41 | 34.74 | 50.66 | 66.20 | 75.26 |
| 1000 ng/mL | | | | | | |

SEM:

| | | | | | | |
|---|---|---|---|---|---|---|
| Control | 0.00 | 1.86 | 2.64 | 2.55 | 4.21 | 5.02 |
| Thymosin alpha 1 | 0.00 | 2.75 | 3.62 | 3.67 | 4.46 | 5.33 |
| 100 ng/mL | | | | | | |
| Thymosin alpha 1 | 0.00 | 1.94 | 2.83 | 5.56 | 5.95 | 7.04 |
| 1000 ng/mL | | | | | | |

TABLE 3
Mean elongation in % compared to day 1

| | Day 0 | Day 1 | Day 3 | Day 5 | Day 7 | Day 9 |
|---|---|---|---|---|---|---|
| Control | 0.00 | 17.38 | 34.92 | 62.57 | 80.50 | 94.86 |
| Thymosin beta4 | 0.00 | 12.26 | 35.49 | 49.79 | 61.36 | 74.11 |
| 100 ng/mL | | | | | | |
| Thymosin beta4 | 0.00 | 14.20 | 39.31 | 66.20 | 90.23 | 103.17 |
| 1000 ng/mL | | | | | | |

SEM:

| | | | | | | |
|---|---|---|---|---|---|---|
| Control | 0.00 | 1.86 | 2.64 | 2.55 | 4.21 | 5.02 |
| Thymosin beta4 | 0.00 | 1.34 | 2.00 | 2.16 | 2.13 | 2.11 |
| 100 ng/mL | | | | | | |
| Thymosin beta4 | 0.00 | 2.30 | 2.13 | 1.67 | 3.22 | 3.63 |
| 1000 ng/mL | | | | | | |

Results obtained in Assay 2 are shown in Tables 4 to 7 and in FIG. 5.

TABLE 4
Mean elongation in % compared to day 0:

| | Day O | Day 3 | Day 5 | Day 7 | Day 9 |
|---|---|---|---|---|---|
| Control | 0 | 21.01 | 32.30 | 41.90 | 48.79 |
| Thymulin 10 pg/mL | 0 | 20.86 | 34.97 | 43.26 | 58.90 |
| Thymosin beta4 | 0 | 19.35 | 31.66 | 41.77 | 49.94 |
| 100 ng/mL | | | | | |
| Thymosin beta4 | 0 | 21.68 | 32.59 | 37.48 | 43.30 |
| 1000 ng/mL | | | | | |

SEM:

| | Day 1 | Day 3 | Day 5 | Day 7 | Day 9 |
|---|---|---|---|---|---|
| Control | 0 | 1.45 | 2.33 | 3.54 | 4.00 |
| Thymulin 10 pg/mL | 0 | 2.16 | 2.38 | 2.26 | 2.81 |
| Thymosin beta4 | 0 | 1.58 | 1.60 | 1.60 | 2.02 |
| 100 ng/mL | | | | | |
| Thymosin beta4 | 0 | 1.47 | 1.72 | 1.55 | 2.22 |
| 1000 ng/mL | | | | | |

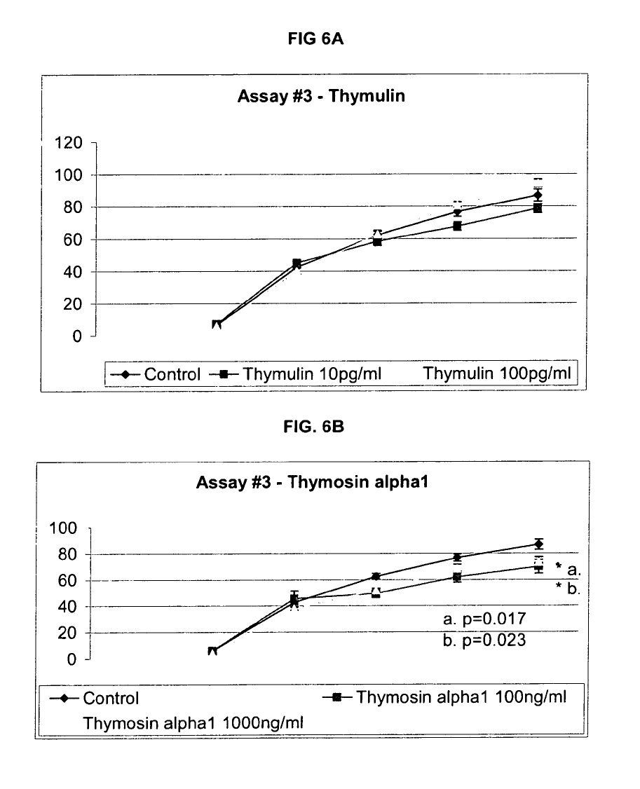

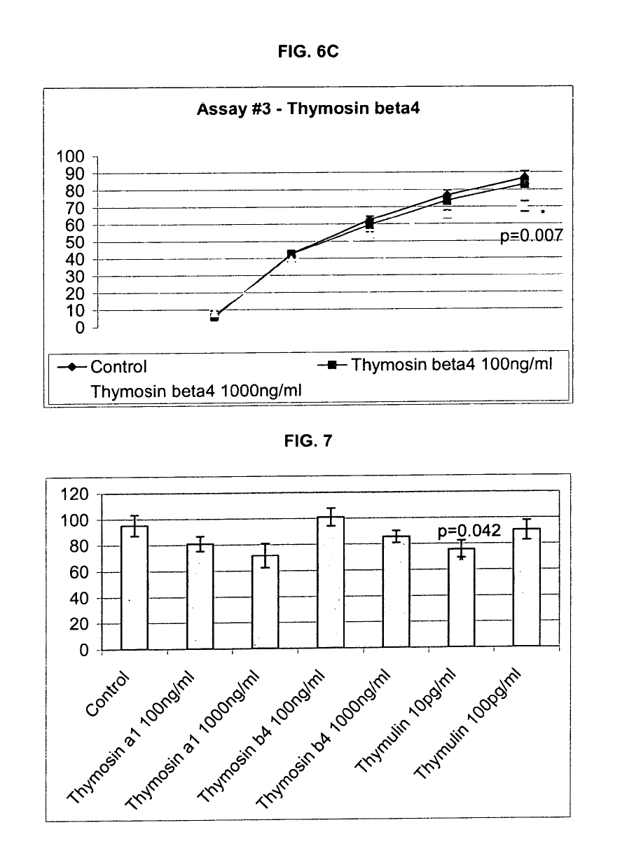

The mean elongation in percent compared to day 0 in assay #3 obtained by thymulin, TA1 and TB4 are shown in Table 4 and FIGS. 6A-6C.

TABLE 4
Mean elongation in % compared to day 0

| | Day 0 | Day 1 | Day 3 | Day 5 | Day 7 | Day 9 |
|---|---|---|---|---|---|---|
| Control | 0 | 6.23 | 42.68 | 62.47 | 76.66 | 86.77 |
| Thymulin 10 pg/mL | 0 | 7.30 | 45.35 | 58.41 | 67.70 | 78.70 |
| Thymulin 100 pg/mL | 0 | 5.86 | 40.11 | 62.88 | 80.67 | 94.36 |
| TA1 100 ng/mL | 0 | 6.25 | 45.55 | 49.52 | 61.94 | 69.90 |
| TA1 1000 ng/mL | 0 | 4.00 | 37.95 | 51.77 | 68.36 | 73.33 |
| TB4 100 ng/mL | 0 | 5.91 | 42.55 | 59.58 | 73.62 | 83.13 |
| TB4 1000 ng/mL | 0 | 8.28 | 39.17 | 53.52 | 65.51 | 70.08 |

SEM:

| | | | | | | |
|---|---|---|---|---|---|---|
| Control | 0 | 0.59 | 1.55 | 2.17 | 2.82 | 3.76 |
| Thymulin 10 pg/mL | 0 | 0.70 | 1.57 | 2.39 | 2.63 | 2.73 |
| Thymulin 100 pg/mL | 0 | 0.95 | 2.05 | 2.31 | 2.19 | 2.37 |
| TA1 100 ng/mL | 0 | 0.80 | 5.95 | 3.16 | 4.09 | 5.25 |
| TA1 1000 ng/mL | 0 | 0.54 | 1.11 | 1.73 | 3.26 | 3.94 |
| TB4 100 ng/mL | 0 | 0.66 | 1.23 | 2.13 | 2.36 | 2.28 |
| TB4 1000 ng/mL | 0 | 1.06 | 1.16 | 1.92 | 2.32 | 3.03 |

2. Treatment with Thymulin Leads to Significantly More Melanin (Assay #1)

Micro-dissected anagen VI hair follicles were treated as above described. At the end of the culture period Fontana Masson staining was performed and photos were taken of the snap frozen sections. The pictures were analysed by measuring the darkness (brightness) of two squares, consisting of 150×150 dots, placed over the darkest areas on both sides of the dermal papilla (dark=small index number, light=high index number). The shading of the different groups was compared to each other and the results were analysed using Student's t-test. The results are shown below in Table 5 and FIG. 7.

TABLE 5
Mean relative darkening of the groups (measured by image])

| | Mean | SEM |
|---|---|---|
| Control | 95.26 | 8.10 |
| Thymosin α1 100 ng/mL | 81.01 | 5.57 |
| Thymosin α1 1000 ng/mL | 71.61 | 9.36 |
| TB4100 ng/mL | 101.04 | 6.75 |
| TB41000 ng/mL | 85.52 | 4.53 |
| Thymulin 10 pg/mL | 75.73 | 6.68 |
| Thymulin 100 pg/mL+ | 90.48 | 7.68 |

Statistical analysis shows that there was significantly more staining in the thymulin 10 pg/mL-treated group than in the control group. A mean darkness (brightness) value of 75.73 indicates therefore that this portion contained relative more melanin pigment.

### 3. Influence of Thymic Hormones on Hair Cycle Stage: Thymulin and TA1 Seem to Keep HFs in Anagen (Assays #1, #2 and #3)

Photodocuments from the Fontana Masson staining were used for hair follicle staging according to previously well-defined morphological criteria (Stenn K S et al, Controls of Hair Follicle Cycling; Physiol Rev 2001, 81:449-494, Müller-Röver S et al, A Comprehensive Guide for the Accurate Classification of Murine Hair Follicles in Distinct Hair Cycle Stages; J Invest Dermatol 2001, 117:3-15). The percentage of hair follicles in anagen, early, mid and late catagen was determined in the different assays. Comparing the three different assays it is evident that groups treated with Thymulin 10 pg and 100 pg/mL have consistently a higher proportion of HFs in anagen than control groups.

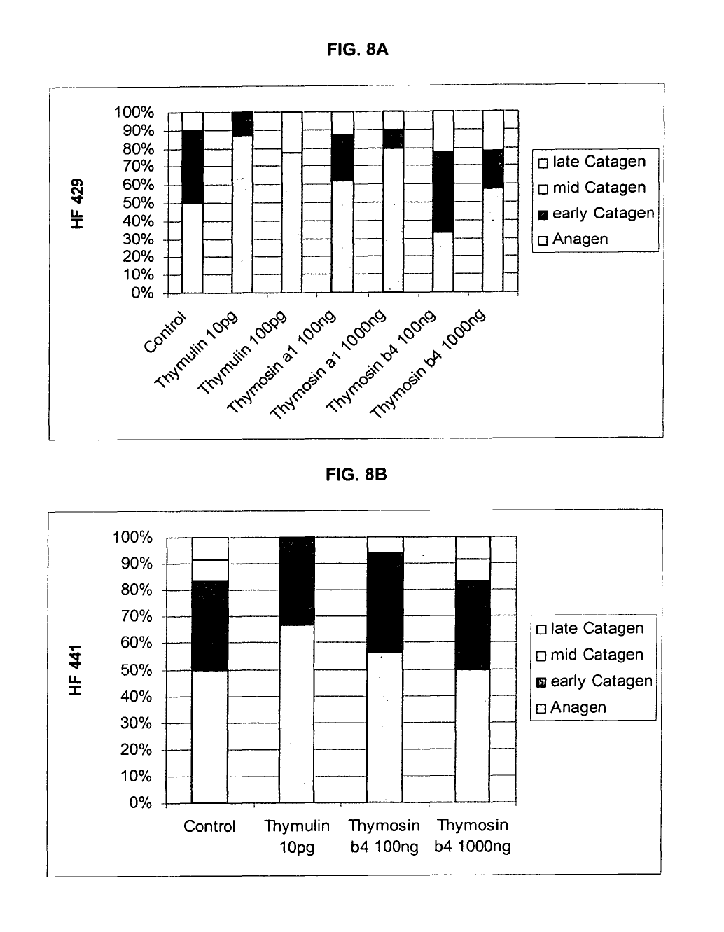

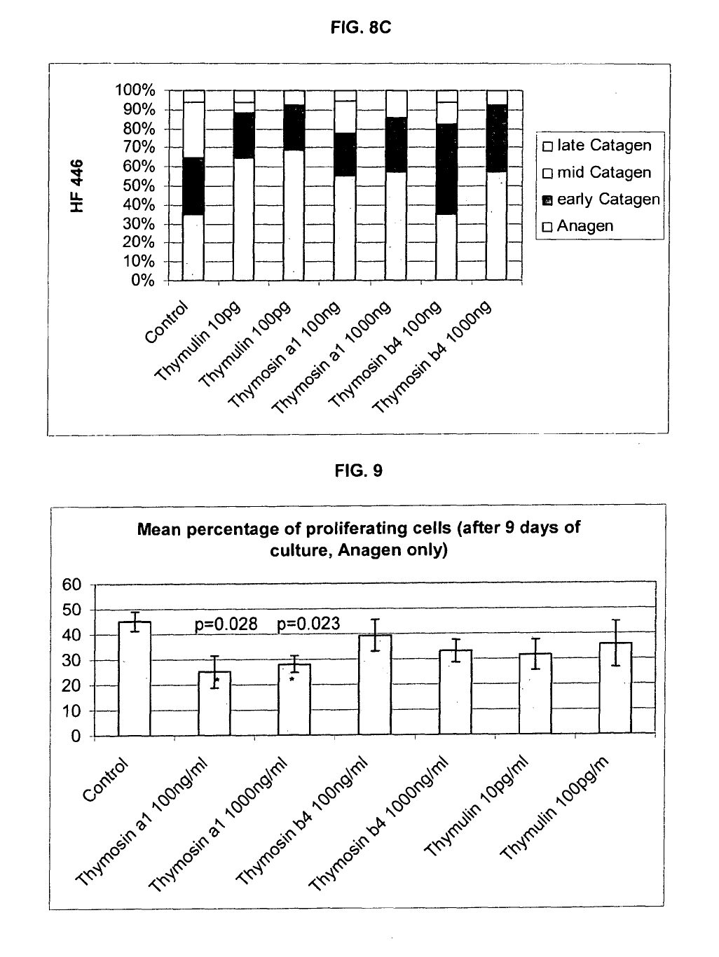

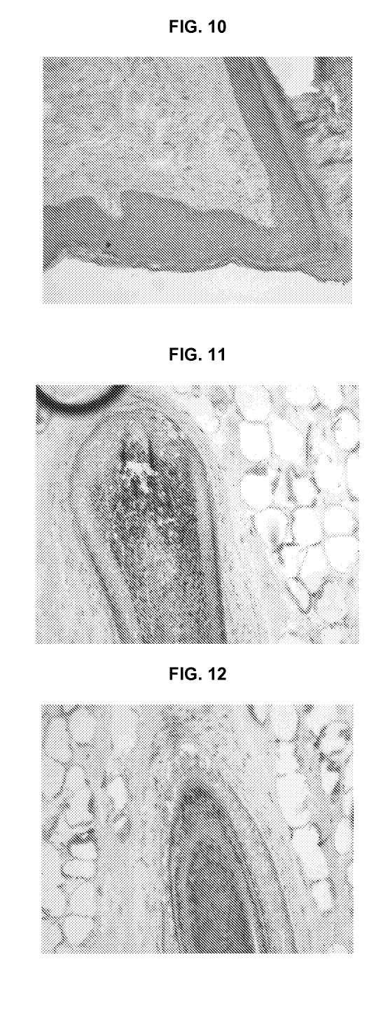

Groups treated with TB4100 ng have in both assays tested (Assay #1 and #3) a higher proportion of cells in catagen compared to control. The proportions of hair follicles in different stages of the hair cycle are shown in FIGS. 8a-c. It should be noted that anagen is the stage of hair growth in the hair cycle, that hair follicles cease to grow for a short period when they reach the stage of catagen (marked by morphological changes that accompany this change) and that they then enter the stage of telogen, which is the resting stage of the hair follicle. Once the hair follicle enters the stage of catagen the growth of hair is finished and it will fall out. Consequently, FIGS. 8a-c show the percentage of hair follicles in the different stages of human hair cycle after a 9-day treatment with the three substances in two concentrations each. The results therefore support that in all three assays there were more follicles in both thymulin-treated and both thymosin alpha-1 treated groups in anagen than in the control groups. This shows therefore that both thymulin and thymosin alpha-1, by keeping human hair follicles longer in the stage of anagen, prevent the hair follicle from entering the stage of catagen and that both thymulin and thymosin alpha-1 can therefore prevent from hair loss.

4. More Precisely, Human Hair Follicles Treated for Nine Days with Thymosin α1 100 ng/mL and 1000 ng/mL Exhibited a Significantly Reduced Percentage of Proliferating Cells Compared to Control (Assay #3)

After nine days, Ki67/TUNEL staining was performed and photos were taken. In anagen hair follicles, proliferating cells and cells undergoing apoptosis were counted separately proximal Auber's line. Results are expressed as percentages. Statistical analysis show a significant (p<0.05) less percentage of cells are proliferating in the groups treated with thymosin-α1 100 ng/mL and thymosin-α1 1000 ng/mL compared to the control. The group with hair follicles treated for two days only proved to be too small to make a statistical analysis. The results are summarized in Table 6 and FIG. 9 showing the mean percentage of Ki6 7 positive cells with SEM

TABLE 6
Mean percentage of proliferating cells
(after 9 days of culture, Anagen only)

| | Mean percentage | |
|---|---|---|
| | of Ki67-positive | |
| | cells | SEM |
| Control | 45.12 | 3.84 |
| TA1100 ng/mL | 25.15 | 6.35 |
| TA11000 ng/mL | 28.05 | 3.33 |
| TB4100 ng/mL | 39.30 | 6.20 |
| TB41000 ng/mL | 33.14 | 4.45 |
| Thymulin 10 pg/mL | 31.58 | 6.00 |
| Thymulin 100 pg/mL | 35.75 | 9.04 |

### 5. Immunohistochemical Staining of Thymic Hormones in Human Scalp Skin and Human Hair Exhibits Stainings in a Specific Looking Pattern (Assay #4)

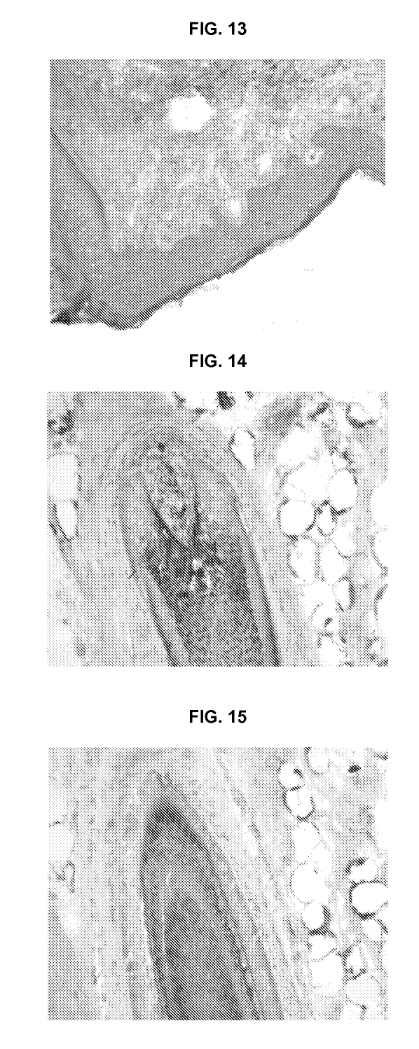

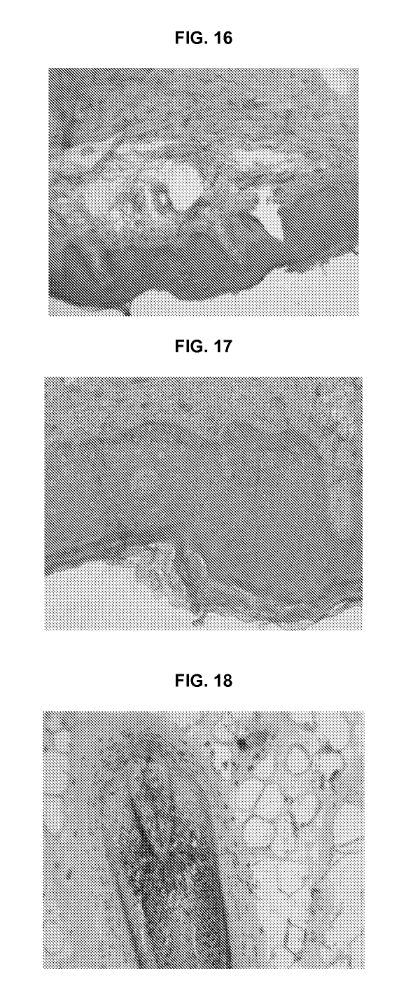

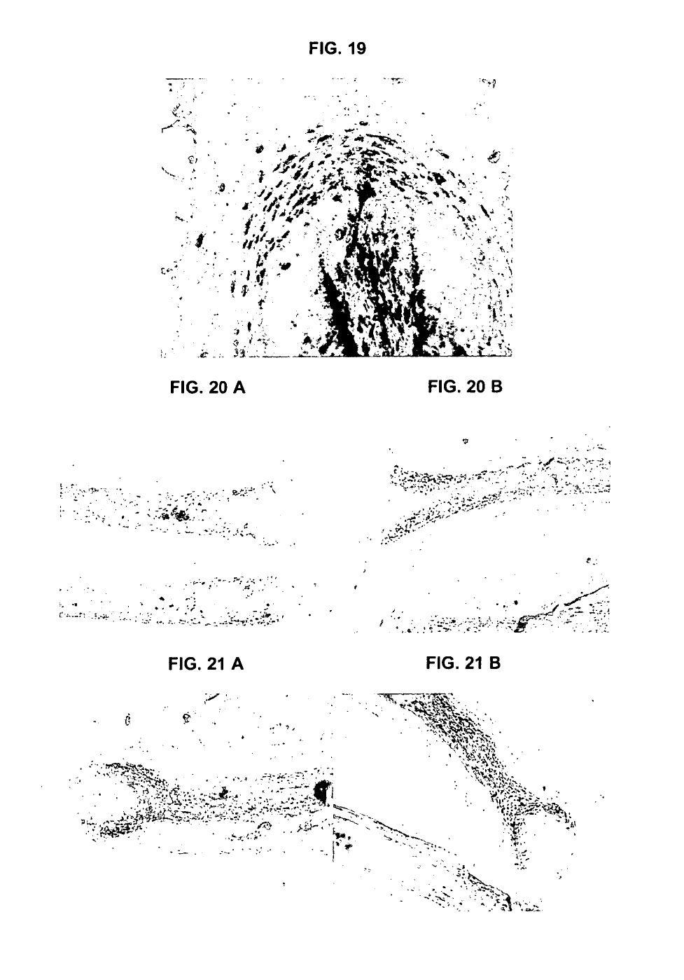

As evident in the pictures, all antibodies to thymic hormones seem to find binding places in the human skin in a specific pattern. Thymulin staining is most impressive in the epidermis, especially close to the basal membrane (FIG. 11). Regarding the hair follicle, the inner root sheath, especially Henle's layer is stained. However, medulla, cortex and outer root sheath (minimal, close to IRS) have also acquired some staining (FIG. 12 and FIG. 13). It is noticeable that the staining starts where the hair bulb merges into the shaft, the matrix cells are not stained. The staining for thymosin alpha1 follows the same pattern: strong staining in the epidermis, strongest at the basal membrane (FIG. 14) and staining of the IRS beginning where the bulb ends (FIG. 15 and FIG. 16). There is little staining of medulla and cortex but no staining of the ORS (contrary to anti-Thymulin). The staining for TB4 stains more ubiquitary than the previous two. There is heavy staining of the dermis, especially the epidermis close to the basal membrane (FIG. 17) in a cytoplasmatic pattern (FIG. 18). In the hair the staining is strongest in the IRS but is present also in ORS, medulla, dermal sheath and dermal papilla (FIG. 19). Interestingly the only place with no staining is the area of matrix cells in the bulb (FIG. 20), an area not stained with anti-Thymulin and anti-thymosin alpha 1, either.

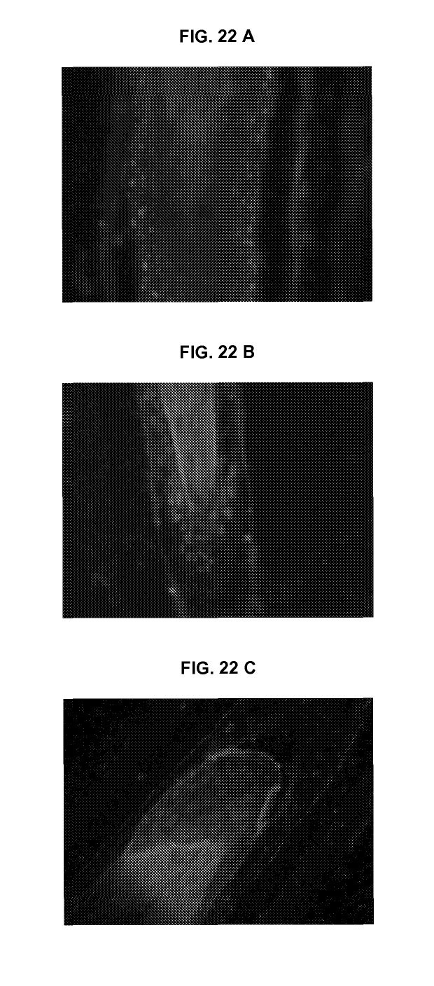

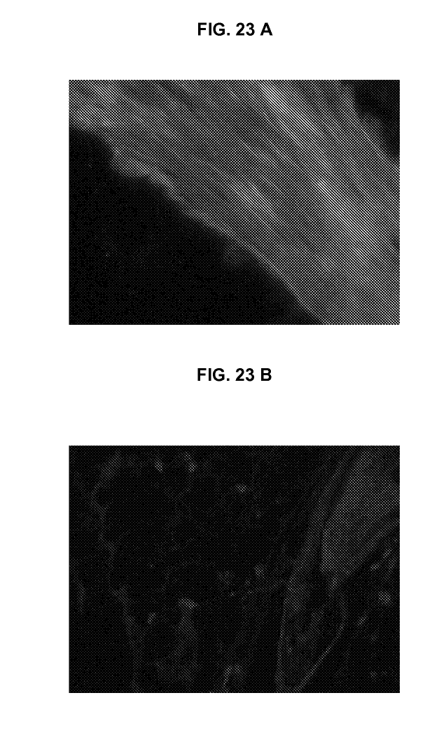

FIG. 22 A-C show immunohistological examinations of the scalp and hair bulb for endogenous thymosin-like peptides with or without acetone fixation of the object and using different antisera for thymosin peptides. FIG. 22A shows an immunostaining of the hair shaft for thymosin-like peptides using a polyclonal anti-thymosin alpha 1 antibody from rabbit (Immundiagnostik AG, Bensheim, DE-Ab CatNo. 9510); FIG. 22B of the inner root sheath using a site-specific anti-thymosin alpha 1 antibody against amino acids 75-109 (Immundiagnostik AG, Bensheim, DE-Ab CatNo. 9540); FIG. 22C of the inner root sheath using a site specific anti-thymosin alpha 1 antibody against amino acids 101-109 (Immundiagnostik AG, Bensheim, DE-Ab CatNo. 9570) FIG. 23A, B show a section and histological staining of human dermis and epidermis using anti-thymosin alpha 1 antibodies. The immunohistological examinations contain abundant evidence that thymosin alpha 1 or at least thymosin-like peptides have a physiological role in the control of the hair growth and cycle.

### 6. Thymulin May Stimulate Human Hair Follicles to Produce Pituitary Hormones (Assay #6)

These very preliminary results indicate that Thymulin might stimulate the hair follicles to produce Prolactin but there is no indication that this is true for TSH, too. However, the slides, which were the “lower quality” slides with partly incomplete follicles from the other tests, suffered visibly from the initial culturing and the following staining method. The immunoreactivity for Prolactin in the untreated follicle was in the area in between IRS and ORS, in keeping with previous findings (Foitzik K et al: Human Scalp Hair Follicles Are Both a Target and a Source of Prolactin, which Serves as an Autocrine and/or Paracrine Promoter of Apoptosis-Driven Hair Follicle Regression; Am J Pathol 2006, 168:748-756). In few hair follicles stimulated with Thymulin 10 pg/nl and 100 pg/nl there was some additional staining in the IRS.

### The Immunoreactivity for TSH Did not Show any Differences in Between Thymulin-Treated and Control Hair Follicles.

Again one must stress, that these investigations were made with only few slides, to few to risk a statement about whether Thymulin is able to stimulate the production of pituitary hormones in hair follicles.

7. ELISA Showed that Thymulin and TA1 are Present in Micro-Dissected Human Hair Follicles (Assay #5)

Thymulin was detected in two of the three samples. The third sample was invalid.

Sample #5a invalid
Sample #5b 0.157 ng/mL
Sample #5c 0.089 ng/mL TA1 was detected in all three samples with the following concentrations:
Sample #5a 2.604 ng/mL (extrapolated data) Sample #5b 5.003 ng/mL
Sample #5c 0.493 ng/mL (extrapolated data)
Thymosin β4: all samples were invalid
Note: Samples #5a and #5b were from the same individual

### Summary

These studies showed that all three substances have different influences on human hair follicles in vitro, while there are interindividual differences in the HF response to thymic hormones.

Thymulin did show different results regarding hair shaft elongation in all three assays. It seemingly tended to elongate the hair shaft in assay #2 and inhibited elongation significantly in assay #1. The content of melanin was significantly raised in the HFs treated with thymulin 10 pg/mL. Highly interesting is the fact that there are consistently more hair follicles in the hair cycle stage of anagen after treatment with thymulin than in the control groups. This result was consistent in all three individuals tested. In these anagen-hair follicles the thymulin-treated groups showed a lower percentage of proliferating cells than control (only one individual tested), but not to a significant level. Immunohistochemistry for thymulin showed staining mainly in the IRS (Henle's layer), medulla and cortex with sparing of the hair matrix cells and dermal papilla. The ORS shows only minimal staining. The human scalp skin showed staining mainly in the epidermis, strongest at the basal membrane. These tests indicate that thymulin stimulates human hair follicles to express prolactin. We could not show the same for TSH. ELISA showed that the human hair follicle contains thymulin.

Thymosin alpha1 inhibited in both assays hair shaft elongation in either concentration, in assay #3 on significant level (both concentrations). We could not find any influence on melanogenesis with the here applied methods. After 9 days of treatment there were more hair follicles in anagen in the thymosin alpha1-groups than in control, this applied to both tested individuals and all concentrations. On examination of these anagen follicles there were significantly less cells proliferating than in the control group (both concentrations). Interestingly, we noticed exactly the same trend with the thymulin-treated groups. Staining for Thymosin alpha1 showed a picture similar to the one for thymulin-staining of the epidermis, strongest close to the basal membrane. The hair follicle shows the heaviest staining in the IRS beginning with the hair shaft and there is no staining of hair matrix cells, dermal papilla and ORS (contrary to thymulin). The ELISA confirmed the presence of thymosin alpha1 in the human hair follicle.

TB4 inhibited hair growth significantly in two out of the six organ cultures. This happened with both concentrations tested but not consistently throughout all tests. Indeed in Assay #1 there was some increased elongation in the follicles treated with a concentration 1000 ng/mL, but not to a significant level. However, the same concentration showed in Assay #3 significantly delayed elongation. We could not find any influence on melanogenesis, hair cycle stage and amount of proliferating cells in our assays with the methods applied. Thymosin β4-treated cells did not show any massive differences in hair cycle stage in our assays, but it is noticeable, that there are consistently more cells in the stage of anagen in the group treated with 1000 ng/mL after nine days in the than in the controls. Hair follicles in anagen, treated with either of the both concentrations of TB4 had less proliferating cells than control but not to the significant level. Staining for TB4 showed heavy staining of the dermis, especially the epidermis close to the basal membrane in a cytoplasmatic pattern. In the hair the staining is strongest in the IRS but is present also in ORS, medulla, dermal sheath and dermal papilla. The only place with no staining is the area of matrix cells in the bulb. The ELISA failed with both assays.

Even though we could not confirm a striking and desired effect of the tested thymic peptides on hair shaft elongation in vitro we certainly have proven that all three proteins significantly influence different components of the hair and of the hair cycle. Contrary to our expectations raised by reports from Philp et al. (The FASEB J Express Article doi:10.1096/fj.03-0244fje Published online Dec. 4, 2003) and Melinda K M et al, Thymosin alpha 1 stimulates endothelial cell migration, angiogenesis and wound healing; J Immunol 1998, 160:1001-6) not a single peptide tested improved significantly the elongation of the cultured hair follicles. Indeed, in a few cases there was significant reduction of the growth rate. Overlooking the (very limited) studies it appears that speeding up the elongation is not the mechanism by which the peptides improved hair growth in the previous mentioned studies. A possible explanation for this paradox could lie in the observation that both, thymulin and thymosin alpha1-treated hair follicles seem to prolong the stage of anagen and/or prevent hair follicles from entering catagen. Groups of both peptides with all tested concentrations had in all three assays a higher percentage of hair follicles in anagen than the control group after 9 days of treatment. One has to speculate by which mechanisms this is achieved. We could not find such a striking correlation in between a “higher than in the control group” number of follicles in anagen and Thymosin β4. Interestingly, this is the only one of the tested peptides where such a correlation has been made (Philp D et al, Thymosin beta4 increases hair growth by activation of hair follicle stem cells; The FASEB J Express Article doi:10.1096/fj.03-0244fje Published online Dec. 4, 2003). However, our results don't stand in contrast to these findings as the TB41000 ng/mL-treated group has in all three assays the same or more hair follicles in anagen. The difference to the control group is just not as impressive as with the other peptides. Interestingly, all substances showed a lower share in proliferating cells in these anagen-hair follicles than the control group (only thymosin alpha 1 to a significant level, a fact that could explain the inhibiting effect on elongation.

We have shown an influence of thymulin on the content of melanin in the hair follicle in the one assay investigated. This result might be of significant clinical importance and it is a completely new finding, which has not been described before. It certainly warrants further research to find out about the underlying mechanism. Furthermore additional tests of Melanogenesis should be applied which may disclose further clues about the influence of thymic peptides on Melanogenesis. The ELISA and immunohistochemistry confirmed the presence of thymic peptides in the hair follicle and human scalp skin although it remains to be confirmed that the staining is specific for the tested thymic peptide.

In view of these results, it is therefore obvious to repeat hair cultures with lower concentrations of TB4 (1 ng/mL and 10 ng/mL) and to perform further tests of melanogenesis (such as counting the active melanocytes etc.) on the already collected data and finally to repeat the hair culture with hair from another individual to establish if the influence of Thymulin on melanogenesis can be repeated. Furthermore, it was noted that GH-treated hair follicles stained more intensively for thymulin than the control group as GH stimulates the secretion of thymulin (Timsit J et al, GH and IGF-1 stimulate hormonal function and proliferation of thymic epithelial cells; J Clin Endocrinol Metab 1992, 75:183-8).

1.-15. (canceled)

16. A method for the treatment or prevention of human hair loss and/or hair graying, comprising administering to a patient suffering from hair loss and/or hair graying an effective amount of one or more thymic peptides of the families of thymulin, thymosin alpha-1 and thymosin beta-4, and a pharmaceutical excipient, diluent or carrier.

17. The method as claimed in claim 16, comprising administering an effective amount of thymulin.

18. The method as claimed in claim 16, comprising administering an effective amount of thymosin alpha-1.

19. The method as claimed in claim 16, comprising administering an effective amount of thymosin beta-4.

20. The method as claimed in claim 16, comprising administering effective amounts of thymulin and thymosin alpha-1, and a pharmaceutical excipient, diluent or carrier.

21. The method as claimed in claim 16, wherein the treatment maintains hair follicle cells longer in the anagen phase of the hair cycle.

22. The method as claimed in claim 16, wherein the hair loss is related to hair shedding and telogen effluvium by catagen inhibition.

23. The method as claimed in claim 16, wherein the hair loss is related to chronic telogen effluvium, nonscarring alopecia, alopecia, hair loss, acute hair loss, non-scarring alopecia or balding.

24. The method as claimed in claim 16, wherein the treatment stimulates human hair follicle pigmentation and/or melanin granule production.

25. The method as claimed in claim 16, wherein the effective amount of one or more thymic peptides is administered topically.

26. The method as claimed in claim 25, wherein the peptides are administered in doses having a concentration ranging from about 1 pg/mL to about 1000 ng/mL.

27. The method of claim 25, wherein the peptides are administered in doses having a concentration ranging from about 10 pg/mL to about 100 ng/mL.

28. The method as claimed in claim 16, wherein the effective amount of one or more thymic peptides is administered orally or systemically.

29. The method as claimed in claim 28, wherein the peptides are administered in doses ranging from about 1 pmol/kg to about 1 nmol/kg.

30. A pharmaceutical composition for the treatment and prevention of human hair loss and/or hair greying, comprising an effective amount of one or more thymic peptides of the families of thymulin, thymosin alpha-1 and thymosin beta-4, and a pharmaceutical excipient, diluent or carrier.

31. The pharmaceutical composition as claimed in claim 30, comprising an effective amount of thymulin and thymosin alpha-1.

32. The pharmaceutical composition as claimed in claim 30, for topical application.

33. The pharmaceutical composition as claimed in claim 32, in the form of an hydrogel.

34. The pharmaceutical composition as claimed in claim 30, for oral or systemic administration.

34. A method for diagnosis of telogen effluvium, balding, hair loss or alopecia, comprising contacting a sample of skin or comprising hair follicles with and antibody against thymulin, thymosin alpha-1 or thymosin beta-4, and quantitating the amount of thymulin, thymosin alpha-1 or thymosin beta-4 in said sample.

35. A kit for diagnosis of telogen effluvium, balding, hair loss or alopecia, comprising containers of antibodies against thymulin, thymosin alpha-1 and thymosin beta-4.
